(19) United States
(12) Patent Application Publication
(10) Pub. No.: US 2019/0008445 A1
(43) Pub. Date: Jan. 10, 2019

(54) HAND AND FOOT ASSISTIVE DEVICE

(71) Applicant: SHUI-MU International Co.,Ltd., Taipei City (TW)

(72) Inventor: Jung-Yueh Lo, Taipei City (TW)

(73) Assignee: SHUI-MU International Co.,Ltd., Taipei City (TW)

(21) Appl. No.: 15/917,819

(22) Filed: Mar. 11, 2018

(30) Foreign Application Priority Data
Jul. 10, 2017 (TW) 106123086

(51) Int. Cl. A61B 5/00; A61B 5/1455; A61B 5/01; A61B 5/024; A61B 5/11; A61B 5/103

(52) U.S. Cl. CPC A61B 5/445; A61B 5/14552; A61B 5/01; A61B 5/024; A61B 5/1118; G01C 22/006; A61B 5/6807; A61B 5/1036; A61B 5/746; A61B 5/6806; A61B 5/0022

(57) ABSTRACT

Disclosed is a hand and foot assistive device capable of taking care of a lesion area of a patient's hand or foot. The device includes a carrier and a measurement module. The carrier is in contact with at least one of the patient's hands and feet. The measurement module is installed at the carrier and includes a sensing unit, a processing unit and a communication unit. The processing unit is coupled to the sensing unit and the communication unit. The sensing unit is disposed adjacent to or overlapped with the lesion area. The sensing unit detects the lesion area to selectively generate a sensing signal. The processing unit processes the sensing signal and sends the sensing signal to communication unit, and the communication unit sends the sensing signal to the outside of the carrier. The sensing unit is provided for detecting a humidity change of the lesion area.

### BACKGROUND OF INVENTION

### 1. Field of the Invention

The present invention relates to the field of medical care, in particular to a hand and foot assistive device applicable for healthcare at home or in medical institutes.

### 2. Description of the Related Art

Diabetes patients usually need to monitor their blood glucose concentration for a long term. If the blood glucose concentration is not controlled properly, the patients' hands and feet may be inflamed, and wounds cannot be healed easily. The inflammation of the hands and feet may cause inflamed swelling, increased local temperature, or open wounds. Before an open wound appears in the inflamed area, it is difficult to determine the severity of inflammation visually. Therefore, it is very important to monitor the temperature distribution of a diabetes patient's hands and feet and blood oxygen concentration, so that before the inflamed area of the hands and feet is deteriorated into a wound, the diabetes patient can find out the actual situation and take necessary medical measures timely. In the meantime, the pulse can also be monitored while monitoring the blood oxygen concentration. If the inflammation continues to deteriorate, then the signal intensity of the monitored pulse will be changed. Therefore, it is necessary to find the inflamed area of the hands and feet turning into a wound ahead of time and take necessary medical measures timely.

Since a diabetes patient's foot is deformed due to the inflamed swelling, the patient has to wear diabetic shoes and socks. Such diabetic shoes or socks generally have an elastic silicone material disposed at the insole or the bottom of the sock to scatter the local pressure and prevent the inflammation wound to continue to deteriorate. In the meantime, the diabetic shoes or socks require good ventilation to avoid high humidity and prevent the breeding and growth of bacteria. Therefore, it is very important to monitor the humidity distribution condition of a diabetes patient's hands and feet, so as to find any inflamed area deteriorated into a wound ahead of time and take necessary medical measures.

As to some patients or potential patients suffering from cardiovascular diseases, if the temperature, humidity and blood oxygen concentration of a patient's hand or foot drops suddenly (when compared with those of the patient's corresponding hand or foot position) or the humidity and blood oxygen concentration are changed suddenly, then it may be vascular embolism, vascular rupture, stroke or varicose vein. The comparison of the temperature, humidity and blood oxygen concentration distribution of different hands and feet may provide warning information, so that the medical staffs can take necessary medical measure timely. If all of the temperature, humidity and blood oxygen concentration of each hand or foot are significantly different from the previous measurements, or the performance of the temperature, humidity and blood oxygen concentration of each hand or foot in a specific daytime period or a specific nighttime period are significantly different from the previous measurements, it is also warning information, and medical staffs have to take necessary medical measures timely.

As to diabetes patients or potential diabetes patients, if they can measure the temperature, humidity and blood oxygen concentration distribution of different parts of the sole while measuring the body weight at home or in a hospital, then the patients can find out the potential sole inflammation or lesion site ahead of time.

In view of the aforementioned drawback of the prior art that cannot take necessary medical measures timely, the present invention provides a hand and foot assistive device to overcome the drawback of the prior art.

### SUMMARY OF THE INVENTION

In view of the drawbacks of the prior art, the inventor of the present invention based on years of experience in the related industry to conduct extensive researches and experiments, and finally developed a hand and foot assistive device to overcome the drawbacks of the prior art.

Therefore, it is a primary objective of the present invention to provide a hand and foot assistive device applicable for the physical care at home or in medical institutes. Patients or medical staffs may use the hand and foot assistive device to monitor a temperature change of a patient's upper limb (including an upper arm, a lower arm, a wrist, a palm, a finger, etc) and the patient's lower limb (including a thigh, a calf, an ankle, a sole, a toe, etc). The device assists a patient to find out a potential abnormal hand and foot condition and provides related information of the abnormal condition to a doctor, so that the doctor can give a proper medical treatment to the patient. It is noteworthy that the upper limbs (or hands) and the lower limbs (or feet) are called limbs in short.

Another objective of the present invention is to provide a hand and foot assistive device capable of synchronously or timely compare the distribution situation of the temperature, humidity, blood oxygen, pulse or weight at different positions of the patient's hands and feet to find any local abnormality and provide related information of the abnormal condition to the doctor, so that the doctor can give a proper medical treatment to the patient.

A third objective of the present invention is to provide a hand and foot assistive device capable of assisting patients or medical staffs to obtain a result of comparing the temperature, humidity, blood oxygen, pulse or weight distribution at different positions of a patient's hands or feet in the same time period (or time), a different time period (or time), the same day, or a different day, so as to provide appropriate warning or healthcare.

To achieve the aforementioned or other objectives, the present invention provides a hand and foot assistive device for taking care of a lesion area of a patient's hands and feet, and the hand and foot assistive device comprises: a carrier, for setting at least one of the patient's hands and feet; and a measurement module, installed to the carrier, and having a sensing unit, a processing unit and a communication unit, and the processing unit being coupled to the sensing unit and the communication unit, and the sensing unit being installed adjacent to or overlapped with the lesion area, and the sensing unit detecting the lesion area to selectively generate a sensing signal, and the processing unit processing the sensing signal and transmitting the sensing signal to the communication unit, and the communication unit sending the sensing signal to the outside of the carrier, wherein the sensing unit detects a humidity change of the lesion area.

In an embodiment, the carrier is a sock, and the sensing units are installed at the toe tip, heel, and sole positions of the sock respectively, wherein the hand and foot assistive devices of the left sock and the right socks have a mirror correspondence.

In an embodiment, the carrier is a glove, and the sensing units are installed at the finger tip and palm positions of the glove respectively, wherein the hand and foot assistive devices of the left glove and the right glove have a mirror correspondence.

In an embodiment, the carrier is a weight scale, and the sensing units are installed at different positions of the weight scale corresponsive to the user's toes respectively.

In summation, the present invention measures the sensing signals of a patient's left hand, right hand, left foot, and right foot by the temperature sensor, humidity sensor, blood oxygen sensor and pulse sensor of a measurement module of the carrier to synchronously and timely detect the distribution situations of the temperature, humidity, blood oxygen, pulse or weight at different positions of the patient's hand or feet, so as to find out any local abnormality and further compare the distribution situations of the temperature, humidity, blood oxygen, pulse or weight of the patient's hands and feet in the same day, a different day, the same time, or a different time. By comparing the distribution situations, the invention assists a patient to find a potential abnormality of the patient's hands and feet and also assists a doctor to diagnose the abnormality and provide a proper medical treatment to the patient.

### BRIEF DESCRIPTION OF THE DRAWINGS

FIG. 1 is a schematic block diagram of a hand and foot assistive device in accordance with a first embodiment of the present invention;

FIG. 2 is a schematic view of a hand and foot assistive device in accordance with a second embodiment of the present invention;

FIG. 3 is a schematic view of a hand and foot assistive device in accordance with a third embodiment of the present invention; and

FIG. 4 is a schematic view of a hand and foot assistive device in accordance with a fourth embodiment of the present invention.

### DESCRIPTION OF THE INVENTION

The above and other objects, features and advantages of this invention will become apparent from the following detailed description accompanied by the drawings.

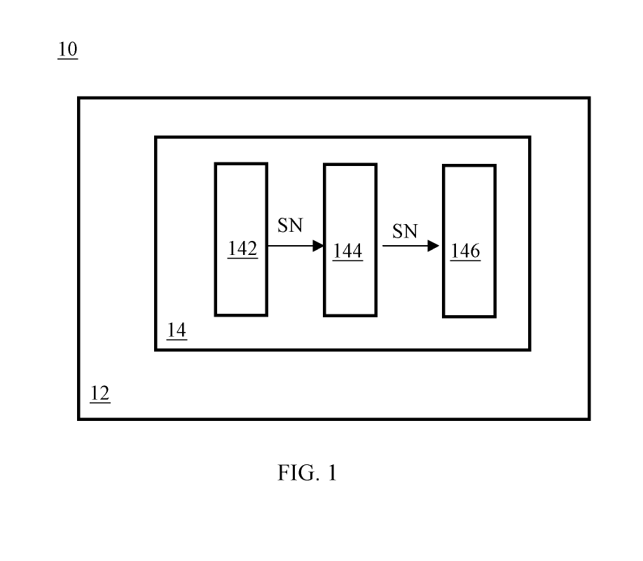

With reference to FIG. 1 for a schematic block diagram of a hand and foot assistive device in accordance with the first embodiment of the present invention, the hand and foot assistive device 10 is capable of providing a warning of a lesion area (such as inflamed swelling, inflammation, fever, wound, etc) of a limb of a patient (not shown in the figure).

The hand and foot assistive device 10 comprises a carrier 12 and a measurement module 14.

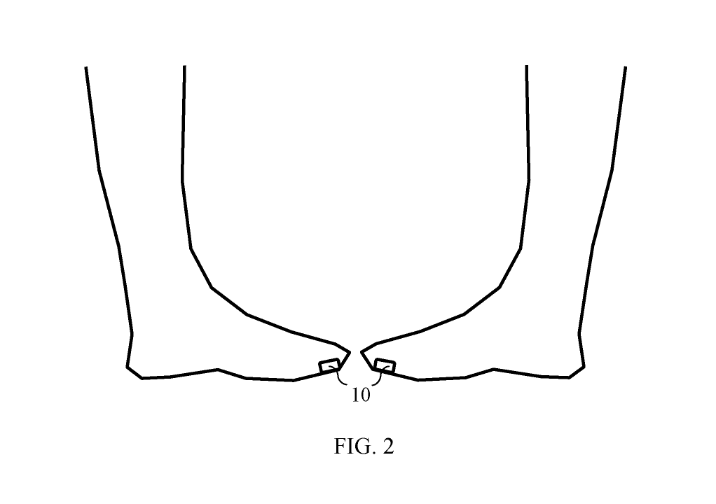

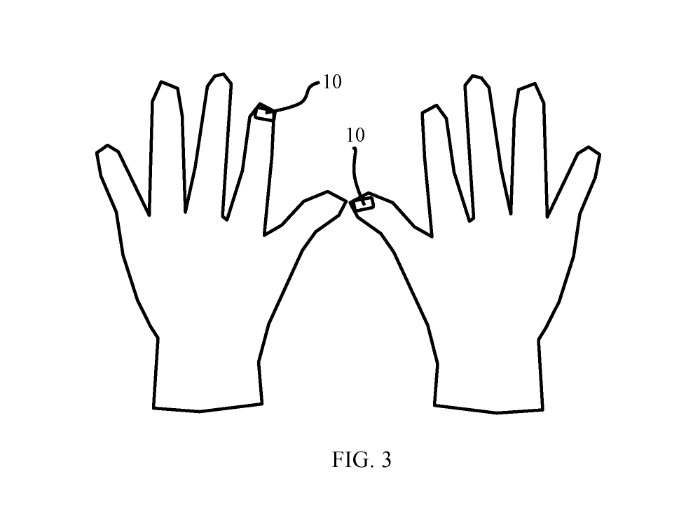

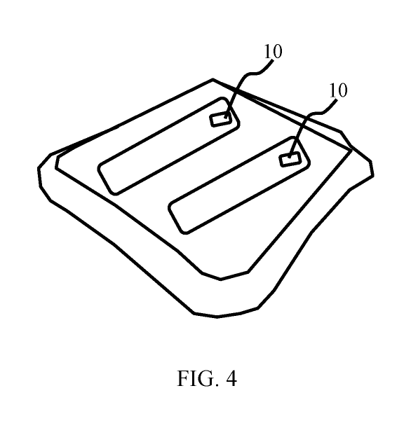

The carrier 12 is provided for touching a patient's limb. For example, the carrier 12 may be a sock, an insole, a shoe, a glove, a weight scale, etc. With reference to FIGS. 2, 3 and 4, a sock, a glove, and a weight scale are used in the embodiments of the present invention respectively.

In FIG. 1, the measurement module 14 is installed to the carrier 12 by an attaching, adhering, buckling, latching, clamping or sewing method. The measurement module 14 further comprises a sensing unit 142, a processing unit 144 and a communication unit 146. The processing unit 144 is coupled to the sensing unit 142 and the communication unit 146.

The sensing unit 142 is disposed adjacent to the lesion area (such as a patient's toe or finger) and provided for precisely detecting a change of the limb. The sensing unit 142 detects the lesion area to selectively generate a sensing signal SN. For example, the sensing unit 142 is a temperature sensor, a blood oxygen sensor, a pulse sensor or a pressure sensor. In this embodiment, the sensing unit 142 detects a humidity change of the lesion area. In another embodiment, the sensing unit 142 also detects a physical quantity such as temperature, pulse, or pressure in addition to the detection of the humidity change. In this embodiment, there is one sensing unit 142. In another embodiment, there are more than one sensing units 142, and the sensing units 142 is installed adjacent to the lesion area.

The processing unit 144 processes the sensing signal SN and sends the sensing signal SN to the communication unit 146. In another embodiment having a plurality of sensing units 142, the processing unit 144 may compare the plurality of sensing signals SN generated by the lesion area detected by the sensing units 142 to determine whether or not to send the sensing signals SN to the outside of the carrier 12 through the communication unit 146, and the carrier may be a mobile phone, a mobile device, a tablet PC, etc.

The communication unit 146 complies with at least one specification selected from the group consisting of a wired communication specification and a wireless communication specification. For example, the wireless communication specification is Bluetooth, wireless fidelity (WiFi), Zig-Bee, etc.

In another embodiment, the hand and foot assistive device 10 further comprises a pedometer unit (not shown in the figure) coupled to the processing unit 144. The pedometer unit is installed at the carrier 12, wherein the pedometer unit may be a gyroscope or a pressure sensor.

In another embodiment, the hand and foot assistive device 10 further comprises a storage unit (not shown in the figure) coupled to the processing unit 144. The storage unit is provided for storing a message of a sensing signal SN (such as a humidity change).

While the invention has been described by means of specific embodiments, numerous modifications and variations could be made thereto by those skilled in the art without departing from the scope and spirit of the invention set forth in the claims.

What is claimed is:

1. A hand and foot assistive device, for taking care of a lesion area of a patient's hands and feet, comprising:
a carrier, for setting at least one of the patient's hands and feet; and
a measurement module, installed to the carrier, and having a sensing unit, a processing unit and a communication unit, and the processing unit being coupled to the sensing unit and the communication unit, and the sensing unit being installed adjacent to or overlapped with the lesion area, and the sensing unit detecting the lesion area to selectively generate a sensing signal, and the processing unit processing the sensing signal and transmitting the sensing signal to the communication unit, and the communication unit sending the sensing signal to the outside of the carrier, wherein the sensing unit detects a humidity change of the lesion area.

2. The hand and foot assistive device of claim 1, wherein the carrier is a sock, an insole, a shoe, a glove or a weight scale.

3. The hand and foot assistive device of claim 1, wherein the sensing unit is one selected from the group consisting of a temperature sensor, a blood oxygen sensor, a pulse sensor and a pressure sensor.

4. The hand and foot assistive device of claim 1, further comprising a pedometer unit installed at the carrier and coupled to the processing unit.

5. The hand and foot assistive device of claim 4, wherein the pedometer unit is one selected from the group consisting of a gyroscope and a pressure sensor.

6. The hand and foot assistive device of claim 1, wherein the communication unit complies with at least one specification selected from the group consisting of a wired communication specification and a wireless communication specification.

7. The hand and foot assistive device of claim 1, further comprising a storage unit coupled to the processing unit for storing a message of the sensing signal.

8. The hand and foot assistive device of claim 1, wherein the sensing unit comes with a plural quantity, and the sensing units are installed adjacent to the lesion area, and the processing unit compares the plurality of sensing signals generated by the lesion area detected by the sensing units to decide to send as least one of the sensing signals to the outside of the carrier through the communication unit.
